SEQUENCE LISTING (9 sequences)

SEQ ID NO: 1 - protein, 271 aa, Homo sapiens
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 2 - DNA, 813 bp, Homo sapiens
gtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcac

SEQ ID NO: 3 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 4 - protein, 29 aa, Homo sapiens
MRALEGPGLSLLCLVLALPALLPVPAVRG

SEQ ID NO: 5 - DNA, 39 bp, Artificial Sequence
  Description of Artificial Sequence oligo primer
gcaccagggtaccaggagctgaggagtgtgagcgtgccg

SEQ ID NO: 6 - DNA, 44 bp, Artificial Sequence
  Description of Artificial Sequence oligo primer
tcagctgcaaggcggcgcgccccgcttgtggtgtcggaccccag

SEQ ID NO: 7 - DNA, 44 bp, Artificial Sequence
  Description of Artificial Sequence oligo primer
ggggtccgacaccacaagcggggcgcgccgccttgcagctgaag

SEQ ID NO: 8 - DNA, 43 bp, Artificial Sequence
  Description of Artificial Sequence oligo primer
gcacagaattcatcagtgcacagggaggaagcgctcacggacg

SEQ ID NO: 9 - DNA, 936 bp, Homo sapiens
  CDS (25)..(924)
gctctccctgctccagcaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcattgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctgggctccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccc

(19) United States
(12) Patent Application Publication
(10) Pub. No.: US 2003/0055221 A1
(43) Pub. Date: Mar. 20, 2003

(54) Stability of flint through o-linked glycosylation

(76) Inventors: Jirong Lu, Indianapolis, IN (US); Derrick Ryan Witcher, Fishers, IN (US)

Correspondence Address: ELI LILLY AND COMPANY, PATENT DIVISION, P.O. BOX 6288, INDIANAPOLIS, IN, 46206-6288, US

(21) Appl. No.: 10/129,709

(22) PCT Filed: Nov. 29, 2000
(86) PCT No.: PCT/US00/30166

(51) Int. Cl. C07K 1/00; C07K 14/00; C07K 17/00

(52) U.S. Cl. 530/350

(57) ABSTRACT

The invention relates to O-linked glycosylated FLINT polypeptides that comprise O-linked oligosaccharides at amino acid position 174 and/or 216 of SEQ ID NO: 1 (i.e. mature FLINT), compositions thereof that may comprise divalent metal cation, clinical and therapeutic uses thereof, and pharmaceutical formulations comprising said polypeptides.

This application claims priority of Provisional Application Ser. No. 60/169,412, filed Dec. 7, 1999.

A number of tumor necrosis factor receptor proteins (“TNFR proteins”) have been isolated in recent years, having many potent biological effects. Aberrant activity of these proteins has been implicated in a number of disease states.

One such TNFR homologue, referred to herein as “Fas Ligand Inhibitory Protein,” or “FLINT”, binds Fas Ligand (FasL), thereby preventing the interaction of FasL with Fas (See WO 99/50413, WO 00/58466, and WO 00/37094, the entire teachings of which are incorporated herein by reference).

Increased activation of the Fas-FasL signal transduction pathway is implicated in a number of pathological conditions, including runaway apoptosis (Kondo et al., Nature Medicine 3(4):409-413 (1997); Galle et al., J. Exp. Med. 182:1223-1230 (1995)), and inflammatory disease resulting from neutrophil activation (Miwa et al.,. Nature Medicine 4:1287 (1998)).

“Runaway apoptosis” is a level of apoptosis greater than normal, including apoptosis occurring at an inappropriate time. Pathological conditions caused by runaway apoptosis include, for example, organ failure in the liver, kidneys and pancreas. Inflammatory diseases associated with excessive neutrophil activation include sepsis, ARDS, SIRS and MODS.

Compounds such as FLINT and analogs thereof, which inhibit the binding of Fas to FasL, and LIGHT to LTβR and/or TR2/HVEM receptors, can be used to treat or prevent diseases or conditions that may be associated with apoptosis and/or inflammation.

The therapeutic utility of FLINT could be enhanced by modifications that improve pharmacological properties (e.g., enhanced potency, stability, longer in vivo half-lives, and/or greater affinity for FasL), pharmaceutical properties (e.g., decreased aggregation and surface adsorption, increased solubility and ease of formulation) and/or chemical properties such as susceptibility to proteolysis. It is known that glycosylation can modulate structure and stability of a protein, influence the activity of signaling molecules, impact molecular recognition, and affect the activity of enzymes (See e.g. P. Van den Steen et al. Critical Reviews in Biochemistry and Molecular Biology, 33(3), 151-208; P. Rudd, Trends in Glycoscience and Glycotechnology, 11, 1-21, 1999).

While FLINT is known to be potentially glycosylated (See e.g. WO 99/50413, WO 99/14330), there is no current understanding as to specific glycosylation patterns nor the impact of specific glycosylation patterns, on the stability of the molecule, or analogs thereof. The therapeutic utility of FLINT is likely to be impacted by the glycosylation pattern. For example, the potency and/or stability of the molecule could be directly impacted by glycosylation, as is known for other proteins. There is therefore a need to understand and to optimize the glycosylation of FLINT and analogs thereof so as to achieve a better, more cost-effective therapeutic protein. The invention disclosed herein relates to a solution to this problem. Specifically, shown herein is that FLINT and analogs produced in mammalian cells have multiple glycosylation species, and that species having O-linked glycosylation are more stable to degradation.

The present invention addresses the need for a FLINT molecule, including analogs thereof, with enhanced stability. Specifically, the invention relates to O-linked glycosylated FLINT polypeptides, N-linked glycosylated FLINT polypeptides, and N/O-linked FLINT polypeptides, and methods for producing and using same. The compositions of the present invention relate further to O-linked glycosylated analogs of FLINT, N/O-linked species, and N-linked glycosylated species of FLINT analogs, wherein one or more complex carbohydrate structures are linked to a threonine, serine, or asparagine residue. The FLINT polypeptides of the invention provide enhanced physical, thermal, and conformational stability to FLINT molecules. The FLINT proteins of the present invention are useful as pharmaceuticals and for treating a variety of diseases and conditions in mammals including humans.

In one embodiment, an oligosaccharide is O-linked to T216 of SEQ ID NO:1 (coincident site T245 of SEQ ID NO:3) and/or T174 of SEQ ID NO:1 (coincident site T203 of SEQ ID NO:3).

In one embodiment, the invention relates to FLINT polypeptides comprising O-linked oligosaccharides.

In another embodiment, the invention relates to FLINT polypeptides comprising O-linked oligosaccharides complexed with a divalent metal cation.

In another embodiment, the present invention relates to FLINT polypeptides comprising O-linked oligosaccharides wherein said oligosaccharides are covalently attached at T216 of SEQ ID NO:1 (T245 of SEQ ID NO:3) and/or T174 of SEQ ID NO:1.

In another embodiment the present invention relates to a FLINT analog comprising N-linked oligosaccharides.

In another embodiment, the invention relates to a FLINT analog polypeptide comprising O-linked and N-linked oligosaccharides wherein said O-linked oligosaccharides are located at T216 of SEQ ID NO:1 (T245 of SEQ ID NO:3) and said N-linked oligosaccharides are located at N144 of SEQ ID NO:1.

In another embodiment, the invention relates to a method for producing a FLINT analog polypeptide comprising O-linked oligosaccharides at T216 of SEQ ID NO:1 said method comprising expression of a vector encoding a protease resistant FLINT analog in a suitable mammalian cell line, for example, AV12, 293 EBNA, or CHO.

In another embodiment, the invention relates to a composition substantially enriched in a FLINT analog polypeptide having O-linked oligosaccharide covalently linked to T216 of SEQ ID NO:1.

In another embodiment the invention relates to therapeutic and clinical uses of a FLINT analog polypeptide substantially comprising O-linked oligosaccharides at T216 to prevent or treat a disease or condition in a mammal in need of such prevention or treatment.

In another embodiment the invention relates to therapeutic and clinical uses of a FLINT analog polypeptide of the present invention to prevent or treat diseases including acute lung injury (ALI), acute respiratory distress syndrome (ARDS), ulcerative colitis, and to facilitate organ preservation for transplantation, to inhibit T lymphocyte activation, to prevent or treat chronic obstructive pulmonary disease (COPD), and to prevent or treat pulmonary fibrosis (PF).

In another embodiment the present invention relates to a pharmaceutical composition substantially comprising an T216 and/or T174/T216 O-linked glycosylated FLINT analog polypeptide of the invention.

In another embodiment, the present invention relates to a method to produce a FLINT analog resistant to proteolysis between position 218 and 219 of SEQ ID NO:1, alternatively between position 247 and 248 of SEQ ID NO:3, comprising the step of increasing O-linked glycosylation at position T216 of SEQ ID NO:1 (alternatively, T245 of SEQ ID NO:3).

In another embodiment, the invention relates to a protease resistant FLINT analog produced by the method of enhancing O-linked glycosylation at position T216 of SEQ ID NO:1.

FLINT polypeptide undergoes proteolysis in vivo to produce at least two major peptide fragments. One of the fragments consists of residues 1 through 218 of SEQ ID NO:1 (alternatively residues 1 through 247 of SEQ ID NO:3), termed herein “FLINT metabolite;” the other consists of residues 219 through 271 of SEQ ID NO:1 (alternatively residues 248 through 300 of SEQ ID NO:3). Cleavage at the 218 position in vitro can be achieved when native FLINT (SEQ ID NO:3), or mature FLINT (SEQ ID NO:1), is treated with a trypsin-like enzyme, for example, thrombin, trypsin or other serine protease. Applicants have discovered that amino acid changes at position 218 render the molecule resistant to proteolysis at that position, as described in PCT application WO 00/58466.

In another embodiment, the invention relates to a FLINT analog that is resistant to proteolysis between positions 218 and 219 of SEQ ID NO:1, and/or between positions 247 and 248 of SEQ ID NO:3 in vivo and/or in vitro, said analog having enhanced O-linked glycosylation at T216.

In another embodiment, the invention relates to a FLINT analog having enhanced O-linked glycosylation at T216, said analog comprising a polypeptide that is at least about 95% identical; alternatively at least 96% identical; alternatively at least 97% identical; alternatively at least 98% identical; alternatively still, at least 99% identical with residues 214 through 222 of SEQ ID NO:1 and/or residues 243 through 251 of SEQ ID NO:3.

In another embodiment, the invention relates to a FLINT analog comprising one, two, three, four, five, or more amino acid substitution(s), deletion(s), or addition(s) in the region comprising amino acids 214-222 of SEQ ID NO:1 and/or amino acids 243-251 of SEQ ID NO:3.

The compositions of the present invention relate to O-linked glycosylated species of FLINT, N/O-linked glycosylated species of FLINT, and N-linked glycosylated species of FLINT, wherein one or more complex carbohydrate structures are linked to a threonine, serine, or asparagine residue. In a preferred embodiment, an FLINT polypeptide comprises oligosaccharide O-linked to T174 of SEQ ID NO:1.

In one embodiment, the invention relates to a FLINT fragment, for example, the metabolite fragment comprising O-linked oligosaccharides.

In another embodiment, the invention relates to FLINT fragments, for example, the metabolite fragment, comprising O-linked oligosaccharides said polypeptides complexed with a divalent metal cation.

In another embodiment, the present invention relates to a FLINT fragment, for example, the metabolite fragment, comprising O-linked oligosaccharides wherein said oligosaccharides are covalently attached at T174 of SEQ ID NO:1 (T203 of SEQ ID NO:3).

In another embodiment, the invention relates to a FLINT fragment, for example, the metabolite fragment, comprising O-linked and N-linked oligosaccharides wherein said O-linked oligosaccharides are located at T174 of SEQ ID NO:1 (T203 of SEQ ID NO:3) and said N-linked oligosaccharides are located at N144 of SEQ ID NO:1.

In another embodiment, the present invention relates to a FLINT fragment, for example, the metabolite fragment, comprising N-linked oligosaccharide.

In another embodiment the invention relates to a FLINT fragment, for example, the metabolite fragment, comprising N-linked oligosaccharide at position N144 of SEQ ID NO:1.

In another embodiment, the invention relates to a method for producing a FLINT polypeptide or fragment thereof comprising O-linked oligosaccharides at T174 and/or T216 of SEQ ID NO:1 said method comprising expression of a vector encoding FLINT in a suitable mammalian cell line, for example, AV12, 293 EBNA, or CHO.

In another embodiment, the invention relates to a method for producing a FLINT polypeptide or fragment thereof comprising N-linked and O-linked oligosaccharide at T174, and/or T216, and N-144 of SEQ ID NO:1.

In another embodiment, the invention relates to a composition substantially enriched in a FLINT polypeptide or fragment, for example the metabolite fragment, having O-linked oligosaccharide covalently linked to T174 of SEQ ID NO:1.

In another embodiment, the present invention relates to a composition substantially enriched in a FLINT polypeptide or fragment, for example the metabolite fragment, having N-linked oligosaccharide.

In another embodiment, the invention relates to a composition substantially comprising a FLINT polypeptide or fragment, for example the metabolite fragment, having O-linked oligosaccharide covalently linked to T174 of SEQ ID NO:1, or T216 of SEQ ID NO:1.

In another embodiment the invention relates to therapeutic and clinical uses of a FLINT polypeptide or fragment, for example the metabolite fragment, comprising O-linked oligosaccharides, or N-linked oligosaccharides, or N/O-linked oligosaccharides, to prevent or treat a disease or condition in a mammal in need of such prevention or treatment.

In another embodiment the invention relates to therapeutic and clinical uses of a FLINT polypeptide or fragment, for example the metabolite fragment, of the present invention to prevent or treat a disease, for example, acute lung injury (ALI), acute respiratory distress syndrome (ARDS), ulcerative colitis, and to facilitate organ preservation for transplantation, to inhibit T lymphocyte activation, to prevent or treat chronic obstructive pulmonary disease (COPD), and to prevent or treat pulmonary fibrosis (PF).

In another embodiment the present invention relates to a pharmaceutical composition comprising an N-linked glycosylated FLINT polypeptide or fragment, for example the metabolite fragment, of the invention.

In another embodiment the present invention relates to a pharmaceutical composition comprising an N/O-linked glycosylated FLINT polypeptide or fragment, for example the metabolite fragment, of the invention.

In one aspect, the FLINT polypeptide of the invention has an amino acid sequence of SEQ ID NO:1, modified by:

a) replacing tryptophan at position 53 with aspartic acid;

b) replacing threonine at position 88 with proline;

c) replacing alanine at position 107 with serine, aspartic acid, glutamic acid or threonine;

d) replacing isoleucine at position 110 with threonine or glutamic acid; or

e) replacing proline at position 104 with serine.

In another aspect, the FLINT polypeptide has an amino acid sequence of SEQ ID NO:1, modified by:

a) replacing alanine at position 2 or position 12 with asparagine;

b) replacing proline at position 25, position 38, position 126 or position 171 with asparagine;

c) replacing arginine at position 35 with asparagine;

d) replacing serine at position 37 with asparagine and proline at position 38 with any other naturally occurring amino acid;

e) replacing serine at position 166 with asparagine;

f) replacing leucine at position 172 with asparagine;

g) replacing aspartic acid at position 194 with asparagine;

h) replacing threonine at position 114 with asparagine and proline at position 115 with any naturally occurring amino acid; or

i) replacing arginine at position 218 with asparagine.

In yet another aspect, the FLINT polypeptide has an amino acid sequence of SEQ ID NO:1, modified by:

a) replacing asparagine at position 63 with tryptophan;

b) replacing glycine at position 67 with aspartic acid and replacing alanine at position 94 or glycine at position 95 with tyrosine;

c) replacing arginine at position 69 with glutamic acid;

d) replacing arginine at position 82 with glutamic acid or threonine;

e) replacing alanine at position 94 with tyrosine and replacing glycine at position 95 with aspartic acid;

f) replacing phenylalanine at position 96 with glutamine;

g) replacing alanine at position 101 with threonine; or

h) replacing glycine at position 95 with aspartic acid.

In yet another aspect, the FLINT polypeptide has an amino acid sequence of SEQ ID NO:1, modified by:

a) replacing arginine at position 10 with glutamine, asparagine, serine or threonine, provided that when the replacing amino acid is asparagine, then alanine at position 12 is optionally replaced with serine or threonine;

b) replacing glutamic acid at position 13 with glutamine, asparagine, serine or threonine, provided that when the replacing amino acid is asparagine, then glycine at position 15 is optionally replaced with serine or threonine;

c) replacing glutamic acid at position 16 with glutamine, asparagine, serine or threonine, provided that when the replacing amino acid is asparagine, then leucine at position 18 is optionally replaced with serine or threonine;

d) replacing arginine at position 17 with glutamine, asparagine, serine or threonine, provided that when the replacing amino acid is asparagine, then valine at position 19 is optionally replaced with serine or threonine;

e) replacing arginine at position 31 with glutamine, asparagine, serine or threonine, provided that when the replacing amino acid is asparagine, then cysteine at position 33 is optionally replaced with serine or threonine;

f) replacing arginine at position 34 with glutamine, asparagine, serine or threonine, provided that when the replacing amino acid is asparagine, then aspartic acid at position 36 is optionally replaced with serine or threonine;

g) replacing arginine at position 35 with glutamine, asparagine, serine or threonine;

h) replacing aspartic acid at position 36 with glutamine, asparagine, serine or threonine, provided that when the replacing amino acid is asparagine, then proline at position 38 is optionally replaced with serine or threonine;

i) replacing arginine at position 143 with glutamine, asparagine, serine or threonine, provided that when the replacing amino acid is asparagine, then cysteine at position 145 is optionally replaced with serine or threonine; or

j) replacing aspartic acid at position 161 with glutamine, asparagine, serine or threonine, provided that when the replacing amino acid is aspargine, then leucine at position 163 is optionally replaced with serine or threonine.

In yet another embodiment, the FLINT of the present invention is a polypeptide having the amino acid sequence of SEQ ID NO:1 modified by:

a) replacing alanine at position 2, 12, 107, 179 or 209 with threonine;

b) replacing threonine at position 4 or 162 with alanine;

c) replacing valine at position 1 or isoleucine at position 110 with methionine;

d) replacing glutamic acid at position 13 with aspartic acid;

e) replacing arganine at position 17 with tryptophan;

f) replacing alanine at position 75 with proline;

g) replacing serine at positione 102 with leucine;

h) replacing glycine at position 169 with alanine;

i) replacing glutamic acid at position 183 with lysine;

j) replacing glutamine at position 225 with arginine;

k) replacing glycine at position 237 with glutamic acid; or

l) replacing valine at position 270 with glycine, said fragment comprising amino acids 49-165 of the polypeptide; and physiologically acceptable salts thereof.

In yet another aspect, the FLINT polypeptide has an amino acid sequence of SEQ ID NO:1, modified by:

a) replacing alanine at position 12 with asparagine and optionally replacing glutamic acid at position 13 with glutamine;

b) replacing arginine at position 34 with asparagine and replacing aspartic acid at position 36 with threonine;

c) replacing arginine at position 35 with asparagine and optionally replacing serine at position 37 with threonine;

d) replacing serine at position 132 with asparagine and optionally replacing serine at position 134 with threonine;

e) replacing aspartic acid at position 194 with asparagine and optionally replacing serine at position 196 with threonine;

f) replacing arginine at position 35 and aspartic acid at position 194 with asparagine;

g) replacing alanine at position 12 with asparagine, optionally replacing glutamic acid at position 13 with glutamine, replacing aspartic acid at position 194 with asparagine and optionally replacing serine at position 196 with threonine;

h) replacing arginine at position 34 with asparagine, replacing aspartic acid at position 36 with threonine, replacing aspartic acid at position 194 with asparagine and optionally replacing serine at position 196 with threonine;

i) replacing arginine at position 35 and aspartic acid at position 194 with asparagine and replacing serine at position 37 and/or position 196 with threonine; or

j) replacing arginine at position 218 with glutamine.

k) replacing glycine at position 26 with aspartic acid and replacing serine at position 132 with asparagine;

l) replacing alanine at position 12 with asparagine, replacing serine at position 132 with asparagine, and replacing serine at position 134 with threonine; or

m) replacing threonine at position 216 with proline and replacing arginine at position 218 with glutamine.

O-linked, and/or N-linked, and/or N/O-linked glycosylated species of FLINT can be produced in mammalian cells transfected with an expression vector that encodes FLINT. Applicants have observed variation in the glycosylation pattern of FLINT molecules produced thereby, for example, in the fraction of polypeptides that are O-linked glycosylated at T174. The fraction of such species may vary depending upon cell culture conditions and on the particular cell type used as host. In a preferred embodiment, a FLINT analog is expressed in CHO cells, from which about 40% of the FLINT polypeptide produced is glycosylated at T174.

### DETAILED DESCRIPTION OF THE INVENTION

SEQ ID NO:1—Mature human FLINT, i.e. native FLINT minus the leader sequence.

SEQ ID NO:2—Nucleic acid/cDNA encoding mature human FLINT.

SEQ ID NO:3—Native human FLINT.

SEQ ID NO:4—Human FLINT leader sequence.

SEQ ID NO:5—Oligonucleotide primer A, CF107

SEQ ID NO:6—Oligonucleotide primer B, CF111

SEQ ID NO:7—Oligonucleotide primer C, CF112

SEQ ID NO:8—Oligonucleotide primer D, CF110

SEQ ID NO:9—Nucleic acid encoding human FLINT.

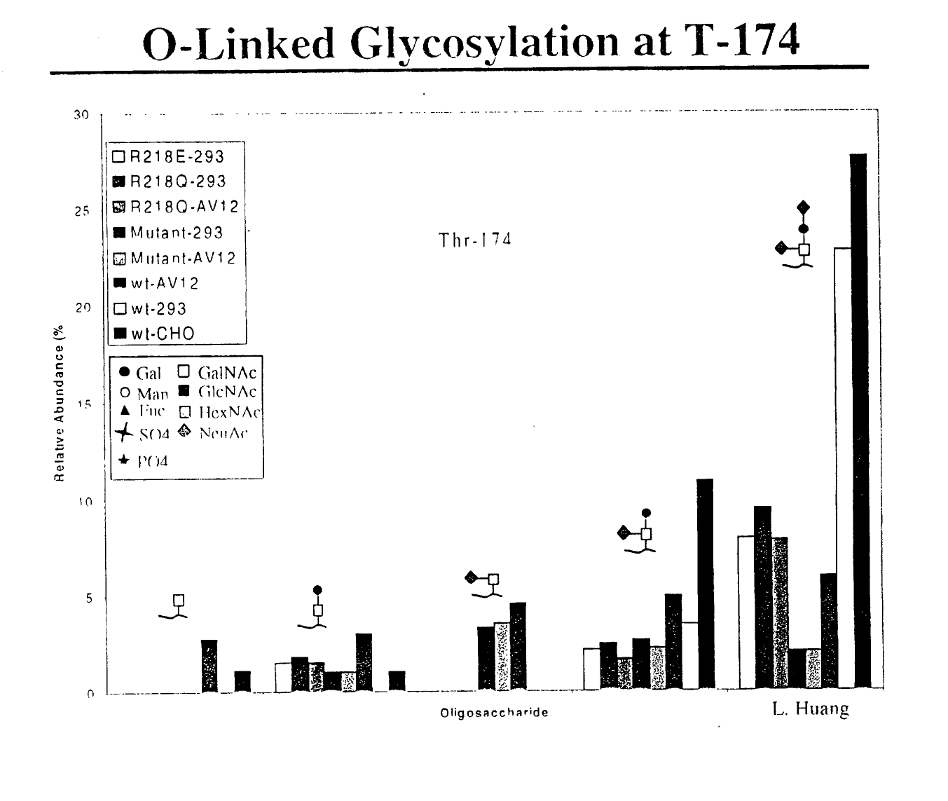

FIG. 1. Oligosaccharide structures at T174 of native FLINT.

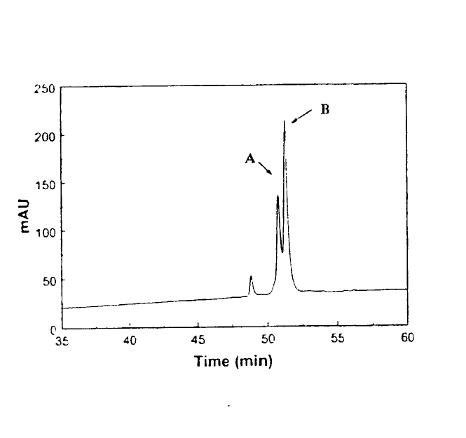

FIG. 2. Reverse phase chromatographic analysis of native FLINT glycosylation variants from AV12 cell line showing both N-linked glycosylation at Asn 144 and O-linked glycosylation at Thr174 (peak A) and N-linked only (peak B).

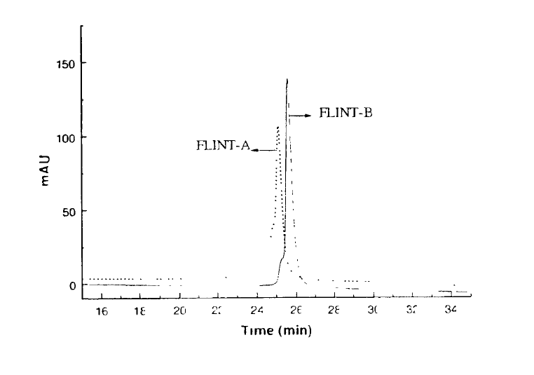

FIG. 3. Reverse phase HPLC chromatogram of FLINT-A (N/O-linked native FLINT) and FLINT-B (N-linked native FLINT).

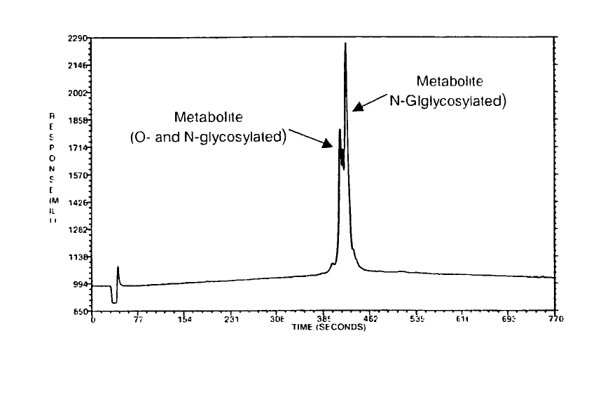

FIG. 4. Reverse phase HPLC chromatogram of FLINT metabolite purified after thrombin cleavage of wt FLINT using a C4 Vydac column.

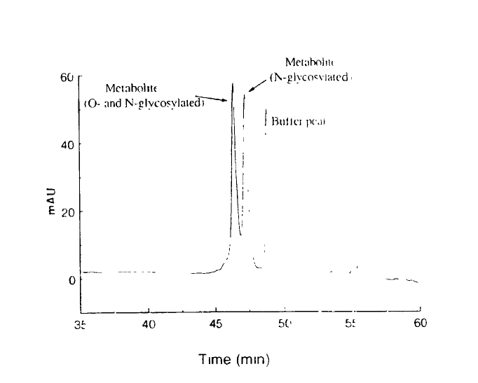

FIG. 5. RP-HPLC chromatogram of FLINT metabolite after concentration step.

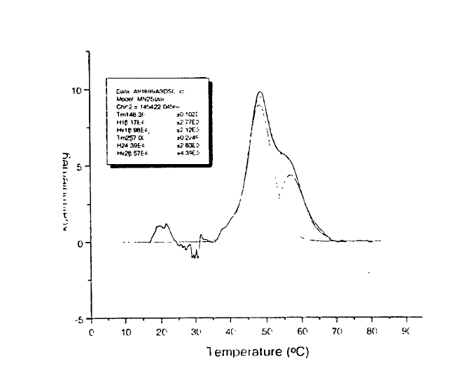

FIG. 6. Thermal denaturation of FLINT metabolite monitored by differential scanning calorimetry. The sample containing 0.2 mg/mL FLINT metabolite was dialyzed against PBS at pH 7.2.

The term “analog” or “FLINT analog” is used herein specifically to mean a FLINT molecule or sequence variant thereof having one or more amino acid sequence changes, e.g. substitution, addition, deletion, including variants that are resistant to proteolysis between positions 218 and 219 of SEQ ID NO:1 and/or positions 247 and 248 of SEQ ID NO:3.

The term “native FLINT” refers to SEQ ID NO:3.

The term “mature FLINT” refers to SEQ ID NO:1.

The term “FLINT” is used herein generically to encompass native and mature FLINT, FLINT fragments, and analogs of FLINT having an altered amino acid sequence comprising one or more amino acid substitutions, deletions, or additions.

The term “FLINT metabolite” or “metabolite” refers to a fragment of FLINT comprising residues 1 to 218 of SEQ ID NO:1.

The term “N-glycosyled polypeptide” refers to polypeptides having one or more NXS/T motifs in which the nitrogen atom in the side chain amide of the asparagine is covalently bonded to a glycosyl group. “X” refers to any naturally occurring amino acid residue except proline. The “naturally occurring amino acids” are glycine, alanine, valine, leucine, isoleucine, proline, serine, threonine, cysteine, methionine, lysine, arganine, glutamic acid, asparatic acid, glutamine, asparagine, phenylalanine, histidine, tyrosine and tryptophan. N-Glycosylated proteins are optionally O-glycosylated. The term “O-glycosyled polypeptide” refers to polypeptides having one or more serines and/or threonine in which the oxygen atom in the side chain is covalently bonded to a glycosyl group. O-Glycosylated proteins are optionally N-glycosylated.

The term “substantially pure” or “substantially enriched” or “substantially comprising” is used herein to mean greater than 50%, preferably 80%-85%; most preferable at least 85% pure, i.e. separated from other proteins and/or other FLINT glycosylation species, and/or compounds and impurities. For example, a pharmaceutical composition substantially comprising FLINT having O-linked glycosylation would refer to a composition comprising a preparation of FLINT in which greater than 50%, preferably 80%-85%, more preferably at least 85% of said FLINT comprised O-linked glycosylated species.

The nucleotide and amino acid abbreviations used herein are those accepted in the art and by the United States Patent and Trademark Office, as set forth in 37 C.F.R. 1.822 (b)(2).

Descriptions herein relating to carbohydrate content of a FLINT polypeptide at position T174 (O-linked), T216 (O-linked), and N144 (N-linked) of SEQ ID NO:1 (mature FLINT) are intended also to relate to SEQ ID NO:3 (native FLINT having leader sequence), wherein the position of carbohydrate content is at 203, 245, and 173, respectively.

Applicants have discovered that mutations in FLINT that render the molecule resistant to proteolysis between the arginine residue at position 218 and the alanine residue at position 219 of SEQ ID NO:1, concomitantly increase the O-linked glycosylation at T216.

Therefore, the invention further contemplates amino acid changes in the region from about position 214 through position 222 of SEQ ID NO:1 or the comparable region of SEQ ID NO:3 that increase the glycosylation at T216.

In one embodiment, a single amino acid change is made within the region 214-222; alternatively, at least two changes are made within this region; alternatively, at least three changes are made within this region; alternatively, at least four changes are made within this region.

In one embodiment, the invention relates to a method for producing a FLINT analog that is resistant to proteolysis between positions 218 and 219 of SEQ ID NO:1 (positions 247 to 248 of SEQ ID NO:3), said method comprising the step of enhancing or increasing O-linked glycosylation at position T216 and/or T174 of SEQ ID NO:1 (T245 and T203 of SEQ ID NO:3 respectively). Said enhancement or increase of O-linked glycosylation can be achieved by changing the cell line in which a recombinant FLINT is expressed. For example, the level of O-linked glycosylation of native FLINT was found to decrease in the order CHO >293 >AV12. Alternatively, enhancement can be achieved by changing the conditions under which a recombinant cell line expressing FLINT is grown. Alternatively still, O-linked glycosylation at T216 can be enhanced by changing the arginine residue at position 218 to a neutral or negatively charged residue. Moreover, changing arginine, a positively charged amino acid, to a neutral or negatively charged amino acid, increases the O-linked glycosylation at T216. For example, R218E is O-linked glycosylated at T216 to a greater extent than R218Q, which, in turn, is O-linked glycosylated at T216 to a greater extent than native FLINT (data not shown).

Applicants have observed variation in the glycosylation pattern of FLINT molecules produced in different cell lines, for example, in the fraction of native FLINT polypeptides that are O-linked glycosylated at T174 and in the composition of oligosaccharides at these positions (See FIG. 1). In a preferred embodiment, FLINT is expressed in CHO cells from which about 50% of the FLINT polypeptide produced is N/O-linked glycosylated at T174. Other cell lines such as AV12 and 293 produced less than 50% N/O-linked species.

O-linked, N-linked, and N/O-linked FLINT polypeptides can be separated from each other and from other glycosylation species of FLINT by a variety of suitable purification techniques, for example, reverse phase HPLC and polyacrylamide gel electrophoresis. Different glycosylation species of FLINT can be resolved by a variety of analytical and preparative techniques. For example, different glycosylation species of FLINT prepared from a mammalian cell line, for example, transfected AV12 cells, can be separated by PAGE or RP-HPLC revealing at least two peaks (See FIG. 2).

Preferred O-linked FLINT polypeptides are glycosylated at T174 and/or T216. O-linked glycosylated FLINT of the invention may also be N-linked glycosylated at position N144. FLINT polypeptides that are expressed in mammalian cells such as 293 EBNA, AV12, and CHO are substantially glycosylated at N144, and partially O-linked glycosylated at T174 and T216. The fraction of FLINT molecules that are O-linked glycosylated at T216 tends to be less than at T174.

The O-linked glycosylated FLINT polypeptides of the invention comprise a plurality of O-linked oligosaccharide structures (See FIG. 1). Predominant oligosaccharide structures at T216 and T174 on FLINT include GalNAc, Galactose, and NeuAc.

Stability of O-linked FLINT Molecules

It is generally known that glycosylation can effect the stability of protein molecules. The invention described herein relates, in one embodiment, to FLINT polypeptides having increased stability, said polypeptides having O-linked oligosaccharides at T174 and/or T216, and N-linked oligosaccharides at N144. Preferably, said FLINT is O-linked glycosylated at T174 and/or T216.

In another embodiment, the invention provides a compound substantially comprising T216 O-linked glycosylated FLINT, alternatively an N-linked FLINT, wherein said glycosylation occurs at, for example, T174 and/or T216, complexed with a divalent metal cation. In another embodiment, the invention provides a compound comprising an N-linked FLINT protein, for example, N144 complexed with a divalent metal cation. In another embodiment, the invention provides a compound comprising an N/O-linked FLINT protein, for example, N144/T174, N144/T174/T216, or N144/T216, complexed with a divalent metal cation.

In another aspect, pharmaceutical compositions comprising O-linked, or N-linked, or N/O-linked glycosylated FLINT and divalent cation(s) are provided. These compositions may be used in depot formulations for therapeutic application.

According to this embodiment, FLINT compositions comprise O-linked, or N-linked, or N/O-linked glycosylated FLINT polypeptides complexed with one or more divalent metal cations. Suitable divalent metal cation include, for example, Zn⁺², Mn⁺², Fe⁺², Co⁺², Cd⁺², Ca⁺², Ni⁺² and the like. Such compositions may comprise a single species of metal ion or a combination of two or more species of divalent metal cations. Preferred compounds comprise a single species of metal cation, most preferably Zn⁺². Preferably, the divalent metal cation is in excess; however, the molar ratio of at least one molecule of a divalent metal cation for each ten molecules of FLINT is operable. Preferably, the compounds comprise from 1 to 100 divalent metal cations per molecule of FLINT. The compounds may be amorphous or crystalline solids.

Appropriate forms of metal cations are any form of a divalent metal cation that is available to form a complex with a molecule of a FLINT protein of the present invention. The metal cation may be added in solid form or as a solution. Several different cationic salts can be used in the present invention. Representative examples of metal salts include the acetate, bromide, chloride, fluoride, iodide and sulfate salt forms. The skilled artisan will recognize that there are many other metal salts which also might be used in the production of the compounds of the present invention. Preferably, zinc acetate or zinc chloride is used to create the zinc-FLINT protein compounds of the present invention. Most preferably, the divalent metal cationic salt is zinc chloride.

The present invention relates further to the use of the FLINT polypeptides of the invention to inhibit apoptosis and/or T cell activation. T cell activation can be chronically suppressed when advantageous, for example, following organ transplantation to prevent rejection, in the treatment of autoimmune diseases, and in treating systemic inflammatory responses.

FLINT polypeptides of the invention can be produced by recombinant techniques or by direct chemical synthesis, well known to the skilled artisan. See. e.g. K. Struhl, “Reverse biochemistry: Methods and applications for synthesizing yeast proteins in vitro,” Meth. Enzymol. 194, 520-535. In a preferred recombinant method, site-directed mutagenesis is used to introduce defined changes into the region 214-222 of SEQ ID NO:1 or the comparable region of SEQ ID NO:3.

FLINT polypeptides also include modified derivatives thereof in which one or more polyethylene glycol groups (hereinafter “PEG” groups) are bonded to the N-terminus or to amine groups or thiol groups in the amino acid side chain(s). Suitable PEG groups generally have a molecular weight between about 5000 and 20,000 atomic mass units. Procedures for preparing PEGylated polypeptides are disclosed in Mumtaz and Bachhawat, Indian Journal of Biochemistry and Biophysics 28:346 (1991) and Franciset al., International Journal of Hematology 68:1 (1998), the entire teachings of which are incorporated herein by reference.

Another embodiment of the invention relates to a fusion protein comprising an O-linked FLINT polypeptide, or an N-linked FLINT polypeptide, or an N/O-linked FLINT polypeptide. Preferred embodiments include T174, T216, T174/T216, N144, T174/N144, T216/N144, T174/T216/N144. “Fusion protein” denotes a hybrid protein molecule not found in nature comprising a translational fusion or enzymatic fusion in which two or more different proteins or fragments thereof are covalently linked on a single polypeptide chain. Human serum albumin and the C-terminal domain of thrombopoietin are examples of proteins which could be fused with a FLINT polypeptide of the invention. Procedures for preparing fusion proteins are disclosed in EP394,827, Tranecker et al., Nature 331:84 (1988) and Fares, et al., Proc. Natl. Acad. Sci. USA 89:4304 (1192), the entire teachings of which are incorporated herein by reference.

A fusion protein of the present invention comprises two protein segments fused together by means of a peptide bond. The first protein segment consists of at least 6, 7, 8, 9, 10, 12, 15, 20, 25, 30, 35, 40, 50, 75, 100, 125, 150, 175, 200, 225, 250, or 271 contiguous amino acid residues of an FLINT polypeptide of the present invention (i.e. derivative of SEQ ID NO:1 or SEQ ID NO:3) having N-linked, or O-linked, or N/O-linked glycosylation; preferably N-linked at N144; O-linked at, alternatively, T174, T216, or T174/T216; and N/O-linked at T174/N144, T216/N144, or T174/T216/N144. The first protein can alternatively be a full length protein of the present invention and/or an N-terminal or C-terminal fragment thereof.

The second protein of a FLINT fusion protein of the invention can be a full length protein or a protein fragment. Proteins commonly used in fusion proteins include B-galactosidase, B-glucuronidase, green fluorescent protein (GFP), thrombopoietin (TPO), glutathione-S-transferase (GST), luciferase, horseradish peroxidase, and chloramphenicol acetyltransferase (CAT). Epitope tags can be used in fusion protein constructions including histidine (His) tags, FLAG tags, influenza hemagglutinin (HA) tags, Myc tags, VSV-G tags, and thioredoxin tags. Other fusion constructions can include maltose binding protein, S-tag, Lex A DNA binding domain, GAL4 DNA binding domain fusions, and herpes simplex virus BP16 protein fusions.

The skilled artisan understands that nucleic acids encoding a FLINT (native or analog) of the present invention can be prepared synthetically. For analogs, this can be achieved by mutating a nucleic acid template that encodes native FLINT, e.g. introducing appropriate point mutations into a cDNA encoding FLINT using any number of suitable mutagenic techniques known to the skilled artisan. Alternatively, said nucleic acids can be prepared synthetically de novo based on knowledge of the genetic code and the particular analog of SEQ ID NO:1 or SEQ ID NO:3 desired. Codon preference may be taken into account when designing a suitable nucleic acid.

A FLINT cDNA can be synthesized by RT-PCR using conventional techniques. For example, PolyA RNA is prepared from a tissue known to express the FLINT gene (e.g. human lung), using standard methods. First strand FLINT cDNA synthesis is achieved in a reverse transcriptase reaction using a FLINT sequence derived “downstream” primer. A commercially available kit such as GENEAMP by Perkin Elmer may be employed. In a subsequent PCR, FLINT specific forward and reverse primers are used to amplify the cDNA. The amplified sample may be analyzed by agarose gel electrophoresis to check the length of the amplified fragment.

FLINT cDNA generated in this manner is used as a template for introducing appropriate point mutations (i.e. construction of FLINT analog cDNAs). A suitable protocol is described in detail in “Current Protocols in Molecular Biology”, volume 1, section 8.5.7 (John Wiley and Sons, Inc. publishers). Briefly, synthetic oligonucleotides are designed to incorporate one or more point mutation(s) at one end of an amplified fragment, e.g. at position 218 of SEQ ID NO:1. Following first strand PCR, the amplified fragments encompassing the mutation are annealed with each other and extended by mutually primed synthesis. Annealing is followed by a second PCR step utilizing 5′ forward and 3′ reverse end primers in which the entire mutagenized fragment gets amplified and is ready for subcloning into the appropriate vector.

The skilled artisan understands that the degeneracy of the genetic code provides multiple codons in some instances for a given amino acid. All such nucleic acid sequence variants are intended to be within the scope of the invention.

Nucleic acid molecules of the present invention can be in the form of RNA, such as mRNA, hnRNA, or any other form, or in the form of DNA, including, but not limited to, cDNA and genomic DNA obtained by cloning or produced synthetically, or any combination thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

“Host cell” refers to any eucaryotic, procaryotic, or other cell or pseudo cell or membrane-containing construct that is suitable for propagating and/or expressing an isolated nucleic acid that is introduced into a host cell by any suitable means known in the art (e.g., transformation or transfection, or the like), or induced to express an endogenous polydeoxynucleic acid. The cell can be part of a tissue or organism, isolated in culture or in any other suitable form.

A variety of eucaryotic expression systems such as yeast, insect cell lines, plant and mammalian cells, are known to those of skill in the art.

Nucleic acid sequences encoding a FLINT polypeptide of the present invention can be ligated to various expression vectors for use in transfecting cell cultures of, for instance, mammalian, insect, or plant origin. Preferred cell cultures useful for the production of FLINT polypeptides are mammalian cells. Mammalian cell monolayers or suspensions may be used. A number of suitable mammalian host cell lines have been developed in the art, including the AV12, 293 EBNA, HEK293, BHK21, and CHO cell lines. Expression vectors for these cell lines can include expression control sequences, such as an origin of replication, a promoter (e.g., the CMV promoter, a HSV tk promoter or pgk (phosphoglycerate kinase) promoter), an enhancer, and processing information sites, such as ribosome binding sites, RNA splice sites, polyadenylation sites (e.g., bovine growth hormone poly A addition site), and transcriptional terminator sequences.

Expression of FLINT in Host Cells

Briefly, the expression of isolated nucleic acids encoding a FLINT analog of the present invention will typically be achieved by operably linking a DNA or cDNA encoding an analog to a promoter (which is either constitutive or inducible), followed by incorporation into an expression vector. The vectors can be suitable for replication and integration in either procaryotes or eucaryotes. To obtain high level expression of a cloned gene, it is desirable to construct expression vectors that contain a promoter to direct transcription, a ribosome binding site for translational initiation, and a transcription/translation terminator. One of skill in the art would recognize that modifications can be made to a protein of the present invention without diminishing its biological activity. For example, some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification handle sequences.

Protein Purification

A FLINT polypeptide of the present invention comprising N-linked, and/or O-linked, an/or N/O-linked oligosaccharides can be recovered and purified from recombinant cells that express said polypeptide by any suitable method, well-known to the skilled artisan including ammonium sulfate or ethanol precipitation, acid extraction, anion or cation exchange chromatography, and immobilized metal ion affinity chelating chromatography, “IMAC,” as taught in U.S. Pat. No. 4,569,974 herein incorporated by reference, phosphocellulose chromatography, hydrophobic interaction chromatography, affinity chromatography, hydroxylapatite chromatography and lectin chromatography. Preferably, species of FLINT polypeptides of the present invention are separated and purified by reverse phase high performance liquid chromatography (“RP-HPLC”).

Therapeutic Applications

FLINT polypeptides inhibit the binding of Fas to FasL and LIGHT to LTβR and TR2/HVEM receptors, and can be used to treat or prevent a disease and/or condition that may be associated with such binding interactions.

FLINT polypeptides of the present invention are clinically and/or therapeutically useful for treating and/or preventing a plurality of diseases including Rheumatoid arthritis (Elliott et al., Lancet 344:1105-10 (1994)), fibroproliferative lung disease, fibrotic lung disease, acute lung injury, acute respiratory distress syndrome, HIV (Dockrell et al., J. Clin. Invest. 101:2394-2405 (1998)), Ischemia (Sakurai et al. 1998 Brain Res 797:23-28), Brain trauma/injury (Ertel et al. 1997 J Neuroimmunol 80:93-6), chronic renal failure (Schelling et al. 1998 Lab Invest 78:813-824), Graft-vs-Host Disease (GVHD) (Hattori et al. 1998 Blood 11:4051-4055), Cutaneous inflammation (Orteu at al. 1998 J Immunol 161:1619-1629), Vascular leak syndrome (Rafi et al. 1998 J Immunol 161:3077-3086), Helicobacter pylori infection (Rudi et al. 1998 J Clin Invest 102:1506-1514), Goiter (Tamura et al. 1998 Endocrinology 139:3646-3653), Atherosclerosis (Sata and Walsh, 1998 J Clin Invest 102:1682-1689), IDDM (Itoh et al. 1997. J Exp Med 186:613-618), Osteoporosis (Jilka et al. 1998 J Bone Min Res 13:793-802), inflammatory bowel disease, ulcerative colitis, Crohn's Disease (van Dullemen et al. 1995 Gastroenterology 109:129-35), organ preservation and transplant (graft) rejection (Lau et al. 1996 Science 273:109-112), Sepsis (Faist and Kim. 1998 New Horizons 6:S97-102), Pancreatitis (Neoptolemos et al. 1998 Gut 42:886-91), Cancer (melanoma, colon and esophageal) (Bennett et al. 1998 J Immunol 160:5669-5675), Autoimmune disease (IBD, psoriasis, Down's Syndrome (Seidi et al., Neuroscience Lett. 260:9 (1999), multiple sclerosis (D'Souza et al. 1996 J Exp Med 184:2361-70), and chronic obstructive pulmonary disease.

An “effective amount” of a FLINT polypeptide is an amount which achieves a desired therapeutic or prophylactic effect in a subject with a disease, or in a method of the invention, that may be associated with aberrant Fas/Fas Ligand binding and/or LIGHT mediated binding. One example of such a process is runaway apoptosis. Alternatively, an “effective amount” of a FLINT polypeptide is a quantity sufficient to achieve a desired therapeutic and/or prophylactic effect in a subject with inflammation caused by Fas Ligand induced neutrophil activation or any of the other aforementioned diseases associated with aberrant Fas Ligand activity.

The amount of FLINT administered to the individual will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors.

As a general proposition, the total pharmaceutically effective amount of FLINT of the present invention administered parenterally per dose will be in the range of about 1 μg/kg/day to 10 mg/kg/day of patient body weight, particularly 2 mg/kg/day to 8 mg/kg/day, more particularly 2 mg/kg/day to 4 mg/kg/day, even more particularly 2.2 mg/kg/day to 3.3 mg/kg/day, and finally 2.5 mg/kg/day, although, as noted above, this will be subject to therapeutic discretion. More preferably, this dose is at least 0.01 mg/kg/day. If given continuously, the FLINT of the present invention are typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing a FLINT polypeptide of the present invention may be administered orally, rectally, intracranially, parenterally, intracisternally, intravaginally, intraperitoneally, topically (as by powders, ointments, drops or transdermal patch), transdermally, intrathecally, bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein includes, but is not limited to, modes of administration which include intravenous, intramuscular, intraperitoneal, intrasternal, subcutaneous and intraarticular injection, infusion and implants comprising FLINT.

For parenteral administration, the FLINT polypeptides of the present invention are formulated generally by mixing at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the FLINT of the present invention uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid: or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The FLINT polypeptides of the present invention are typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of salts of the FLINT analogs of the present invention.

The invention is illustrated by the following examples which are not intended to be limiting in any way.

### EXAMPLE 1

### Production of a Vector for Expressing FLINT Analog R218Q

FLINT variant R218Q was constructed by mutagenic PCR starting from a wild-type FLINT template. See e.g. Saiki R. K. et al. Science 239:487-491 (1988), and “Current Protocols in Molecular Biology”, Vol 1, section 8.5.7 (John Wiley and Sons, Inc. publishers). The R218Q mutant substitutes an arginine residue found at amino acid 218 with glutamine. The mutagenic PCR process utilized a SOEing reaction (i.e. Strand Overlap Extension) to create specific mutations in the native FLINT template for the purpose of changing the amino acid sequence at position 218, and further for introducing restriction enzyme tags for identification purposes. Generally, SOEing reactions require the use of four primers, two in the forward orientation (termed A, SEQ ID NO:5, and C, SEQ ID NO:7) and two in the reverse orientation (termed B, SEQ ID NO:6 and D, SEQ ID NO:8). The SOEing reaction amplifies a nucleic acid sequence (e.g. gene sequence) in two stages. The first step is to amplify “half” the gene by performing an A to B reaction followed by a separate C to D reaction. In constructing the R218Q mutant, the B and C primers were targeted to the same area of the gene but on opposite strands. Mismatch priming from both oligonucleotide primers institutes the mutation. After these two reactions were completed, the products were isolated and mixed for use as the template for the A to D reaction, which yields the desired mutated product. The primers involved in the cloning of R218Q were:

Primer A: CF 107 (39 nt)
GCACCAGGGTACCAGGAGCTGAGGAGTGTGAGCGTGCCG
Primer B: CF 111 (44 nt)
TCAGCTGCAAGGCGGCGCGCCCCGCTTGTGGTGTCGGACCCCAG
Primer C: CF 112 (44 nt)
GGGGTCCGACACCACAAGCGGGGCGCGCCGCCTTGCAGCTGAAG
Primer D: CF 110 (43 nt)
GCACAGAATTCATCAGTGCACAGGGAGGAAGCGCTCACGGACG

Using the forward primer C as a reference, the bold G and C show the silent changes necessary to introduced an AscI site. This recognition site is underlined in primers B and C.

The 311 base pair amplified fragment carrying the R218Q mutation was sub-cloned using a 5′ KpnI site (GGTACC) and a 3′ EcoRI site (GAATTC). The native FLINT sequence has a naturally occurring internal KpnI site around amino acid position 176. The EcoRI site was introduced for sub-cloning purposes and lies downstream of the stop codons. These sites are underlined in primers A and D respectively. The 311 bp fragment was incorporated into the full length FLINT sequence. This was accomplished through the following steps:

The 311 bp fragment was placed into an intermediate vector, pCR2.1- TOPO, which utilizes the adenine overhangs established after PCR for ligation.

Once incorporated, a KpnI to EcoRI digestion removed a 289 bp fragment. (Note: the size of the PCR fragment decreased from 311 bp to 289 bp due to the digestion). The mutated fragment was used to replace the corresponding segment in the wild type FLINT gene by directional ligation.

FLINT/pJB03 was digested with KpnI to EcoRI to produce two fragments

Fragment 1: 6070 bp

Fragment 2: 289 bp

The 6070 bp fragment carrying the FLINT gene was isolated and ligated with the 289 bp PCR product removed from the pCR 2.1-TOPO vector to create R218Q/pJB03. Positive clones were identified by restriction digestion and subsequently confirmed by sequence analysis. The R218Q analog contained in R218Q/pJB03 was shuffled into the pIG3 vector by means of a NheI to XbaI undirected ligation. R218Q/pJB03 was digested with NheI to XbaI to yield fragments of 932 and 5427 bp. The 932 bp FLINT R218Q containing fragment was isolated. The 932 bp NheI and XbaI fragment of R218Q was ligated with the 8510 bp linearized pIC 3 vector to generate clones in both forward and inverse orientations.

### EXAMPLE 2

### Construction of Vector pIG3 for Expression of FLINT in Mammalian Cells

A bicistronic expression vector was constructed by inserting into mammalian expression vector pGTD (Gerlitz, B. et al., 1993, Biochemical Journal 295:131) a PCR fragment encoding an “internal ribosome entry site”/enhanced green fluorescent polypeptide (IRES/eGFP). The new vector, designated pIG3, contains the following sequence landmarks: the Ela-responsive GBMT promoter (D. T. Berg et al., 1993 BioTechniques 14:972; D. T. Berg et al., 1992 Nucleic Acids Research 20:5485); a multiple cloning site (MCS); the IRES sequence from encephalomyocarditis virus (EMCV); the eGFP coding sequence (Cormack, et al., 1996 Gene 173:33, Clontech); the SV40 small “t” antigen splice site/polyadenylation sequences; the SV40 early promoter and origin of replication; the murine dihydrofolate reductase (dhfr) coding sequence; and the ampicillin resistance gene and origin of replication from pBR322.

Based on the human FLINT cDNA sequence (e.g. SEQ ID NO:3), forward and reverse PCR primers were synthesized bearing BclI restriction sites at their respective 5′ends. These primers were used to amplify the FLINT cDNA. The FLINT cDNA orientation and nucleotide sequence was confirmed by restriction digest and double stranded sequencing of the insert. The approximately 900 base pair amplified FLINT analog PCR product was digested with restriction endonucleases NheI and XbaI, respectively, to generate a fragment bearing NheI and XbaI sticky ends. This fragment was subsequently ligated into a unique XbaI site of pIG3 to generate recombinant plasmid pIG3-FLINT. The insert encoding FLINT can be modified at the C-terminus of the analog to introduce a cleavable hexahistidine (His6) cassette to facilitate analog purification.

### EXAMPLE 3

### Isolation of High-Producing FLINT Clone from AV12 RGT18 Transfectants

The recombinant plasmid of Example 2 carries the FLINT gene and encodes resistance to methotrexate. In addition, the construct contains a gene encoding a fluorescent polypeptide, GFP, on the same transcript and immediately 3′ to the FLINT gene. Since high level expression of GFP would require a high level of expression of the FLINT-GFP mRNA, highly fluorescent clones have a greater probability of producing high levels of FLINT.

AV12 RGT18 cells are transfected using a calcium phosphate procedure with recombinant plasmid pIG1. Cells resistant to 250 nM methotrexate are selected and pooled. The pool of resistant clones is subjected to fluorescence assisted cell sorting (FACS), and cells having fluorescence values in the top 5% of the population are sorted into a pool and as single-cells. High fluorescence pools are subjected to two successive sorting cycles. Pools and individual clones from the first and second cycles are analyzed for FLINT production by ELISA. Pools or clones expressing FLINT at the highest level are used for scale-up and FLINT purification.

### EXAMPLE 4

### Large Scale Purification of Native O-linked FLINT Polypeptide

Large scale production of mature FLINT (mFLINT) was carried out by growing stable clones of AV12 RGT 18 cells transfected with pIG3-FLINT in several 10 liter spinners. After reaching confluency, cells were further incubated for 2-3 more days to secrete maximum amount of mFLINT into the growth medium. Medium containing mFLINT was adjusted to 0.1% CHAPS and concentrated in an Amicon ProFlux M12 tangential filtration system to 350 ml. The concentrated media was adjusted to pH 6.0 and passed over a SP Sepharose Fast Flow (Pharmacia, 500 ml) at a flow rate of 7 ml/min. The column was washed with buffer A (20 mM MOPS, 0.1% CHAPS, pH 6.0) until the absorbance (280 nm) returned to baseline and bound polypeptides were eluted with a linear gradient from 0 to 1 M NaCl (in buffer A) developed over four column volumes. Fractions containing mFLINT were pooled and passed over Vydac C4 column (100 ml) equilibrated with 0.1% TFA/H₂0 at a flow rate of 10 ml/min.

The bound mFLINT was separated into a predominantly N-linked species, and a predominantly N/O-linked mFLINT species by elution with a linear gradient 0% to 50% CH₃CN/0.1% TFA over 10 column volumes followed by a linear gradient from 50% to 90% CH₃CN/0.1% TFA over 1 column volumne. Fractions containing N/O-linked mFLINT and N-linked mFLINT were separately pooled, concentrated under vacuum to approximately 4 ml and an equal volume of PBS, 0.5 M NaCl, 10% glycerol, 100 mM Tris, 50 mM EDTA, pH 7.0 was added. The purified samples were dialyzed against four liters of PBS, 0.5 M NaCl, 10% glycerol, 1 mM EDTA, pH 7.4 three times and four liters of PBS, 0.5 M NaCl, 10% glycerol, pH 7.4 three times. Substantially pure fractions obtained in this fashion contain either predominantly N/O-linked or N-linked mFLINT. Analysis by SDS-PAGE revealed these fractions to be greater than 85% pure. The N-terminal sequence of mFLINT was confirmed on the purified polypeptides. LC-MS was performed on the polypeptides to confirm the location of the glycans.

### EXAMPLE 5

### Quantitation of FLINT Analogs

FLINT analogs can be quantitated in crude media of transfected cells and during purification procedure by a developed FLINT ELISA. ELISA uses anti-FLINT polyclonal antibody TKD-028(1494) as a capture antibody and biotinylated anti-FLINT TKD-076A as a primary antibody in a “sandwich” assay. ELISA is developed by streptavidin derivatized horse radish peroxidase (SA-HRP) using OPD as a substrate and monitoring the absorbance at 490 mn. The useful range of such an ELISA is from 0.2-20 ng/ml.

### EXAMPLE 6

### Effect of ZnCl₂ on Thermal Stability of FLINT

The conformational stability of FLINT in the presence or absence of ZnCl₂ was monitored by differential scanning calorimetry (DSC). Data was collected on a VP-DSC MicroCalorimeter using VPViewer software and Origin DSC software for data analysis. The matched sample and reference cells had a working volume of 0.5 mL. FLINT samples were dialyzed against buffer containing 25 mM Tris, 150 mM NaCl at pH 6.8 overnight and the concentration of protein was determined by UV absorbance at 277 nm using extinction coefficient of 0.786 for 1 mg/mL protein sample in 1-cm pathlength cell.. Buffer was also run overnight in both cells to establish a thermal history prior to sample runs. Proteins were then diluted to approximately 0.15 mg/mL, and the dialysate was used as the reference solution. 2 ul or 4 ul of ZnCl₂ at 5 mM was added to 1 mL protein sample to obtain 10 uM ZnCl₂ concentration. After degassing, sample and reference were loaded in cells with 2.5 mL needle through a filling funnel. Pressure was kept at approximately 30 psi. with a pressure cap. Data was collected between 5° and 100° C. The denaturation process was partially reversible. The material was recovered and analyzed by RP-HPLC to determine the ratio of N and O-linked glycosylation species. The fraction of N/O-linked species increased from 49% before thermal denaturation to 55% after denaturation, indicating that material containing O-linked glycosylation is more stable than material containing only N-linked glycosylation.

### EXAMPLE 7

### Large Scale N and O-linked FLINT Analog Polypeptide Purification

Large scale production of FLINT R218Q is carried out by first growing stable pIG1-R218Q-containing AV12 RGT 18 cells in several 10 liter spinners. After reaching confluency, cells are further incubated for 2-3 more days to secrete maximum amount of FLINT analog into media. Medium containing FLINT analog is adjusted to 0.1% CHAPS and concentrated in an Amicon ProFlux M12 tangential filtration system to 350 ml. The concentrated medium is adjusted to pH 6.0 and passed over a SP Sepharose Fast Flow (Pharmacia, 500 ml) at a flow rate of 7 ml/min. The column is washed with buffer A (20 mM MOPS, 0.1% CHAPS, pH 6.0) until the absorbance (280 nm) returns to baseline and the bound polypeptides are eluted with a linear gradient from 0 to 1 M NaCl (in buffer A) developed over four column volumes. Fractions containing FLINT are pooled and passed over Vydac C4 column (100 ml) equilibrated with 0.1% TFA/H₂O at a flow rate of 10 ml/min. The bound FLINT analog is separated into N-linked and O-linked FLINT analog and N-linked FLINT analog by elution with a linear gradient 0% to 50% CH₃CN/0.1% TFA over 10 column volumes followed by a linear gradient from 50% to 90% CH₃CN/0.1% TFA over 1 column volume. Fractions containing N- and O-linked FLINT and N-linked FLINT are pooled separately, concentrated under vacuum to approximately 4 ml and an equal volume of PBS, 0.5 M NaCl, 10% glycerol, 100 mM Tris, 50 mM EDTA, pH 7.0 is added. The purified samples are dialyzed against four liters of PBS, 0.5 M NaCl, 10% glycerol, 1 mM EDTA, pH 7.4 three times and four liters of PBS, 0.5 M NaCl, 10% glycerol, pH 7.4 three times. Fractions containing N- and O-linked versus N-linked FLINT are analyzed by SDS-PAGE. The N-terminal sequence of FLINT is confirmed on the purified polypeptides. LC-MS is performed on the polypeptides to confirm the location of the glycans.

### EXAMPLE 8

### Use of O-linked Glycosylated FLINT to Treat ALI Patient

A 55 year-old male presents to the emergency department unconscious. His family states that he was being treated as an outpatient for bronchitis for the past few days but worsened despite antibiotics. He has no relevant past history and his only medication was a third generation oral cephalosporin. Physical examination reveals an obtunded, cyanotic male who is hypotensive, tachypneic, and tachycardic, and who has bilateral lung congestion consistent with pulmonary edema. There is no evidence of congestive heart failure. Tests reveal hypoxemia (based on PaO2/FiO2) and bilateral lung infiltrates without cardiomegaly, consistent with a diagnosis of acute lung injury. Based on the history it is concluded that the lung injury was a direct result of community-acquired pneumonia, and that the patient met the hypoxemia criteria for ALI within the last 12 hours. Ventilation measures include use of PEEP and low tidal volume. As soon as adequate oxygenation is confirmed, treatment with O-linked glycosylated FLINT is initiated in the ER as an iv bolus of 2.5 mg/kg, followed by a continuous infusion of 0.1 mg/minute. FLINT along with aggressive supportive measures (e.g., positive ventilation, intravenous fluids, pressors, and nutritional support) are continued for four days in the ICU, at which time the FLINT is discontinued. Over the following 3 days, the patient begins to recover and is extubated on Day 8. He has an uneventful recovery and 6 months following discharge has no evidence of residual lung disease by blood gas and spirometry.

### EXAMPLE 9

### Effect of ZnCl₂ on Thermal Stability of FLINT Analog

The conformational stability of FLINT analog R218Q in the presence or absence of ZnCl₂ is monitored by differential scanning calorimetry (DSC). Data is collected on a VP-DSC MicroCalorimeter using VPViewer software and Origin DSC software for data analysis. The matched sample and reference cells have a working volume of 0.5 mL. FLINT samples are dialyzed against buffer containing 25 mM Tris, 150 mM NaCl at pH 6.8 overnight and the concentration of protein determined by UV absorbance at 277 nm using extinction coefficient of 0.786 for 1 mg/mL protein sample in 1 cm path length cell. Buffer is also run overnight in both cells to establish a thermal history prior to sample runs. Proteins are diluted to approximately 0.15 mg/mL, and the dialysate is used as the reference solution. 2 ul or 4 ul of ZnCl₂ at 5 mM is added to 1 mL protein sample to obtain 10 uM and 20 uM ZnCl₂ concentration, respectively. After degassing, sample and reference are loaded in cells with 2.5 mL needle through a filling funnel. Pressure is kept at approximately 30 psi. with a pressure cap. Data is collected between 5° and 100° C. Thermal scan of FLINT analog with or without ZnCl₂ is taken. Data is analyzed to obtain the midpoint of thermal transition (Tₘ). FLINT analog sample contains two populations of molecules, one containing only N-linked glycosylation at Asn144, the other containing both N-linked glycosylation and O-linked glycosylation at T174 and/or T216. These two populations of molecules are separated by RP-HPLC. The data suggest that ZnCl₂ has a stabilizing effect on FLINT analog containing additional O-linked glycosylation compared to FLINT only containing N-linked glycosylation.

### EXAMPLE 10

### Production of the FLINT Metabolite

FLINT was purified from AV12 RGT 18 cells transfected with a recombinant vector carrying a FLINT cDNA (SEQ ID NO:1 or SEQ ID NO:3). This material was cleaved with thrombin at a weight ratio of 1 to 100 (thrombin to FLINT) for three hours at room temperature, dialyzed against 20 mM MOPS, 0.1% CHAPS, pH 6.5, and passed over a SP Sepharose column at a flow rate of 1 ml/min. The column was washed with buffer A (20 mM MOPS, 0.1% CHAPS, pH 6.5) until the absorbance returned to baseline. The bound metabolite (amino acids 1 to 218 of SEQ ID NO:1) was eluted with a linear gradient from 0 to 300 mM NaCl (in buffer A) developed over 10 min. followed by a linear gradient for 0.3 to 0.5 M (in buffer A). Fractions were analyzed by SDS-PAGE and mass spectrometry. Fractions containing only the FLINT metabolite (1-218) were pooled and concentrated in Millipore Ultrafree centrifugal filter. The concentrated metabolite (1-218) was again analyzed by SDS-PAGE and mass spectrometry to assess purity. N-terminal sequencing confirmed the identity of the purified material as FLINT metabolite (1-218).

### EXAMPLE 11

### Improving Stability of FLINT Metabolite Through O-linked Glycosylation

When FLINT was injected subcutaneously or intravenously in mice, a metabolite containing amino acids 1-218 of FLINT was formed. This metabolite can also be generated using thrombin cleavage in vitro.

To investigate the effect of differential N and O-linked glycosylation on the stability of FLINT metabolite, FLINT produced as in Examples 2 through 4 was treated with thrombin and the metabolite produced thereby analyzed by reverse phase HPLC and differential scanning calorimetry As shown in FIG. 4, FLINT metabolite contains two species when analyzed by RP-HPLC, N-linked and N/O-linked. Metabolite containing N/O-linked glycosylation is more stable than N-linked species, as shown by RP-HPLC. As the sample was concentrated in about 200 mM NaCl, 20 mM MOPS buffer using Millipore ultrafree-4 centrifugal filter unit (10,000 MWCO membrane), material containing only N-linked glycosylation was preferentially lost compared to material containing both N-linked and O-linked glycosylation, leading to the change in the relative peak areas for these two species (FIG. 5).

The thermal stability of FLINT metabolite in PBS monitored by differential scanning calorimetry showed two transitions as shown in FIG. 6, with midpoint of transition temperature at 48° C. for the first transition and 57° C. for the second transition. The high melting temperature transition may be attributed to FLINT metabolite containing both N- and O-linked glycosylation. The second transition disappeared with addition of 2 mM EDTA into the sample, suggesting that divalent metal ions are likely to be involved in the stabilization of FLINT metabolite.

The data presented above suggest that additional O-linked glycosylation provides better physical stability to FLINT metabolite. It is conceivable that mutations to introduce additional glycosylation sites or fusion protein of FLINT metabolite with Fc can also be beneficial to the stability of FLINT metabolite.

What is claimed is:

1. A FLINT polypeptide comprising O-linked oligosaccharides.

2. A FLINT polypeptide as in claim 1 wherein said O-linked oligosaccharides occur at Thr 174 of SEQ ID NO:1.

3. A FLINT polypeptide as in claim 1 wherein said O-linked oligosaccharides occur at Thr 216 of SEQ ID NO:1.

4. A pharmaceutical formulation comprising as an active ingredient a FLINT polypeptide according to claim 1 associated with one or more pharmaceutically acceptable carriers, excipients, or diluents thereof.

5. A method for increasing the stability of a FLINT polypeptide comprising the step of enhancing O-linked glycosylation.

6. A method to enhance resistance to proteolysis at position 218 of SEQ ID NO:1 comprising the step of increasing O-linked glycosylation at position 216 of SEQ ID NO:1.

7. A FLINT polypeptide composition substantially comprising a polypeptide of claim 1 and a divalent metal cation.

8. A composition as in claim 14 wherein said cation is selected from the group consisting of Zn⁺², Ca⁺², Ni⁺², Mn⁺², Fe⁺², Co⁺², and Cd⁺².

9. A FLINT polypeptide comprising N-Linked oligosaccharides.
